(19) United States
(12) Patent Application Publication
(10) Pub. No.: US 2010/0305047 A1
(43) Pub. Date: Dec. 2, 2010

(54) CYCLOSPORIN DERIVATIVES FOR ENHANCING THE GROWTH OF HAIR

(75) Inventors: David F. Woodward, Lake Forest, CA (US); Michael E. Garst, Newport Beach, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/785,158

(22) Filed: May 21, 2010

Related U.S. Application Data
(60) Provisional application No. 61/181,388, filed on May 27, 2009.

(51) Int. Cl. A61K 38/13; A61P 17/14

(52) U.S. Cl. 514/20.5

(57) ABSTRACT

The present invention provides a method for the treatment of alopecia and/or enhancing hair growth in a patient, wherein said method comprises administering, preferably topically, to the skin, e.g. the scalp or the eyelid, a therapeutically effective amount of a cyclosporin A derivative selected from the group consisting of compounds represented by the formula:

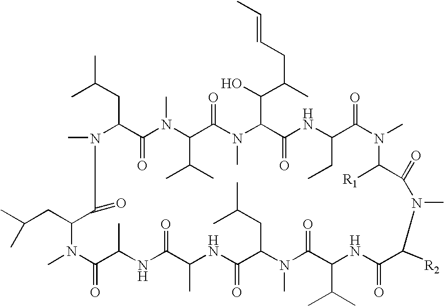

wherein R₁ is S-Alk-R wherein Alk is an alkylene or alkylenyl linkage, R is a hydrogen or a unsubstituted or substituted hydrocarbyl group and R₂ is selected from the group consisting of hydroxyl, lower alkyl and hydroxyl substituted lower alkyl.

### CROSS-REFERENCE

This application claims the benefit of U.S. Provisional Patent Application Ser. No. 61/181,388, filed on May 27, 2009, the entire disclosure of which is incorporated herein by this specific reference.

### BACKGROUND OF THE INVENTION

1. Field of the Invention

The invention relates to a method of enhancing the growth of hair with cyclosporine derivatives and, in particular, the present invention relates to a method for the treatment of alopecia using certain novel cyclosporine derivatives. In particular, this invention relates to a method for stimulating the growth of mammalian hair comprising the topical application to the scalp of a derivative of cyclosporine A, e.g. a novel cyclosporine A derivative, or a pharmacologically acceptable salt thereof, in association with a topical pharmaceutical carrier.

2. Description of the Related Art

Dermatologists recognize many different types of hair loss, the most common by far being “alopecia” wherein human males begin losing scalp hair at the temples and on the crown of the head as they get older. While this type of hair loss is largely confined to males, hence its common name “male pattern baldness,” it is not unknown in women. No known cure has yet been found despite continuing attempts to discover one.

A good deal is known about various types of human hair and its growth patterns on various parts of the body.

For purposes of the present invention, it is necessary to consider various types of hair, including, terminal hairs and vellus hairs and modified terminal hairs, such as seen in eye lashes and eye brows. Terminal hairs are coarse, pigmented, long hairs in which the bulb of the hair follicle is seated deep in the dermis. Vellus hairs, on the other hand, are fine, thin, non-pigmented short hairs in which the hair bulb is located superficially in the dermis. As alopecia progresses, a transition takes place in the area of approaching baldness wherein the hairs themselves are changing from the terminal to the vellus type.

Another factor that contributes to the end result is a change in the cycle of hair growth. All hair, both human and animal, passes through a life cycle that includes three phases, namely, the anagen phase, the catagen phase and the telogen phase. The anagen phase is the period of active hair growth and, insofar as scalp hair is concerned, this generally lasts from 3-5 years. The catagen phase is a short transitional phase between the anagen and telogen phases which, in the case of scalp hair, lasts only 1-2 weeks. The final phase is the telogen phase which, for all practical purposes, can be denominated a “resting phase” where all growth ceases and the hair eventually is shed preparatory to the follicle commencing to grow a new one. Scalp hair in the telogen phase is also relatively short-lived, some 3-4 months elapsing before the hair is shed and a new one begins to grow.

Under normal hair growth conditions on the scalp, approximately 88% of the hairs are in the anagen phase, only 1% in catagen and the remainder in telogen. With the onset of male pattern baldness, a successively greater proportion of the hairs are in the telogen phase with correspondingly fewer in the active growth anagen phase.

Alopecia is associated with the severe diminution of hair follicles. A bald human subject will average only about 306 follicles per square centimeter, whereas, a non-bald human in the same age group will have an average of 460 follicles per square centimeter. This amounts to a one-third reduction in hair follicles which, when added to the increased proportion of vellus hair follicles and the increased number of hair follicles in the telogen phase, is both significant and noticeable. Approximately 50% of the hairs must be shed to produce visible thinning of scalp hair. It is thus a combination of these factors: transition of hairs from terminal to vellus, increased number of telogen hairs—some of which have been shed, and loss of hair follicles that produces “baldness”.

While a good deal is known about the results of male pattern baldness, very little is known about its cause. The cause is generally believed to be genetic and hormonal in origin although, the known prior art attempts to control it through hormone adjustment have been singularly unsuccessful.

One known treatment for male pattern alopecia is hair transplantation. Plugs of skin containing hair are transplanted from areas of the scalp where hair is growing to bald areas with reasonable success; however, the procedure is a costly one in addition to being time-consuming and quite painful. Furthermore, the solution is inadequate from the standpoint that it becomes a practical, if not an economic, impossibility to replace but a tiny fraction of the hair present in a normal healthy head of hair.

Other non-drug related approaches to the problem include such things as ultra-violet radiation, massage, psychiatric treatment and exercise therapy. None of these, however, has been generally accepted as being effective. Even such things as revascularization surgery and acupuncture have shown little, if any, promise.

By far, the most common approach to the problem of discovering a remedy for hair loss and male pattern alopecia has been one of drug therapy. Many types of drugs ranging from vitamins to hormones have been tried and only recently has there been any indication whatsoever of even moderate success. For instance, it was felt for a long time that since an androgenic hormone was necessary for the development of male pattern baldness, that either systemic or topical application of an antiandrogenic hormone would provide the necessary inhibiting action to keep the baldness from occurring. The theory was promising but the results were uniformly disappointing.

The androgenic hormone testosterone was known, for example, to stimulate hair growth when applied topically to the deltoid area as well as when injected into the beard and pubic regions. Even oral administration was found to result in an increased hair growth in the beard and pubic areas as well as upon the trunk and extremities. While topical application to the arm causes increased hair growth, it is ineffective on the scalp and some thinning may even result. Heavy doses of testosterone have even been known to cause male pattern alopecia.

Certain therapeutic agents have been known to induce hair growth in extensive areas of the trunk, limbs and even occasionally on the face. Such hair is of intermediate status in that it is coarser than vellus but not as coarse as terminal hair. The hair is generally quite short with a length of 3 cm. being about maximum. Once the patient ceases taking the drug, the hair reverts to whatever is normal for the particular site after six months to a year has elapsed. An example of such a drug is diphenylhydantoin which is an anticonvulsant drug widely used to control epileptic seizures. Hypertrichosis is frequently observed in epileptic children some two or three months after starting the drug and first becomes noticeable on the extensor aspects of the limbs and later on the trunk and face. (The same pattern of hypertrichosis is sometimes caused by injury to the head.) As for the hair, it is often shed when the drug is discontinued but may, in some circumstances, remain.

Streptomycin is another drug that has been found to produce hypertrichosis, in much the same way as diphenylhydantoin, when administered to children suffering from tuberculous meningitis. About the same effects were observed and the onset and reversal of the hypertrichosis in relation to the period of treatment with the antibiotic leave little question but that it was the causative agent.

Two treatments have been demonstrated as showing some promise in reversing male pattern alopecia. These treatments include the use of a microemulsion cream containing both estradiol and oxandrolone as its active ingredients and the use of organic silicon.

In addition to the foregoing, it has been reported in U.S. Pat. Nos. 4,139,619 and 4,968,812 that the compound minoxidil is useful for the treatment of male pattern baldness. That compound, among others, has proven to have considerable therapeutic value in the treatment of severe hypertension. It is a so-called “vasodilator” which, as the name implies, functions to dilate the peripheral vascular system. Dermatologists and others have recognized that prolonged vasodilation of certain areas of the human body other than the scalp sometimes result in increased hair growth even in the absence of any vasodilating therapeutic agent. For instance, increased hair growth around surgical scars is not uncommon. Similarly, arteriovenous fistula have been known to result in increased vascularity accompanied by enhanced hair growth. Externally-induced vasodilation of the skin, such as, for example, by repeated biting of the limbs by the mentally retarded and localized stimulation of the shoulders by water carries has been known to bring on hypertrichosis in the affected areas. Be that as it may, similar techniques such as continued periodic massage of the scalp have been found to be totally ineffective as a means for restoring lost hair growth to the scalp. Scar tissue on the scalp inhibits rather than promotes hair growth.

U.S. Pat. No. 6,262,105 to Johnstone suggests that prostaglandins and derivatives thereof are useful in a method of enhancing hair growth.

Methylthio-substituted cyclosporin A and other alkylthio-substituted cyclosporin A derivatives have been described in PCT application Nos. 98-379455, 98-379456 and 98-379457 and have been found to be active against certain retroviruses, especially AIDS (acquired immunodeficiency syndrome) and ARC (AIDS-related complex) when administered orally, parenterally, rectally or by inhalation. In addition, they have generally been found to have only a very weak immunosuppressant action, and to show anti-retroviral activity at non-cytotoxic and non-cytostatic concentrations. These compounds are claimed to have a synergistic action with other agents active against retrovirus (such as inhibitors of reverse transcriptase, protease, integrase, HIV replication and nucleocapside).

It is one object of this invention to provide novel cyclosporine A derivatives to treat alopecia.

It is another object of the invention to provide new cyclosporine A derivatives to enhance hair growth.

It is, therefore, a principal object of the present invention to provide a novel and effective treatment for the stimulation of hair growth and the treatment of male pattern baldness.

Another object of the invention is to provide a method of stimulating hair growth in humans and non-human animals that is compatible with various types of therapeutic agents or carriers and, therefore, would appear to be combinable with those which, by themselves, demonstrate some therapeutic activity such as, for example, microemulsion creams or topical compositions containing estradiol and oxandrolone, minoxidil or agents that block the conversion of testosterone to dihydrotesterone (Procipia).

Still another objective is the provision of a treatment for the stimulation of hair growth which, while effective for its intended purpose, is apparently non-toxic and relatively free of unwanted side effects.

An additional object of the invention herein disclosed and claimed is to provide a method for treating hair loss in men or women which can be applied by the patient under medical supervision no more stringent than that demanded for other topically-administered therapeutic agents.

Other objects of the invention are to provide a treatment for male pattern alopecia which is safe, simple, painless, cosmetic in the sense of being invisible, easy to apply and quite inexpensive when compared with hair transplants and the like.

Other objects of this invention will become apparent from a reading of the present specification.

### SUMMARY OF THE INVENTION

The present invention provides a method for treating alopecia, comprising the step of administering to a patient in need thereof, a therapeutically effective amount of a compound selected from the group of cyclosporin A derivatives represented by the below general formula. That is the cyclosporin A derivatives utilized in the method(s) of the present invention are represented by the formula

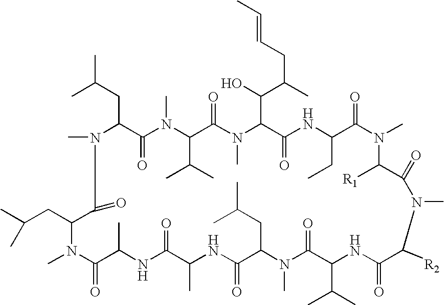

wherein R₁ is S-Alk-R wherein Alk is an alkylene linkage, preferably a methylene or poly methylene linkage, e.g. a C₂ to C₆ polymethylene linkage, or a polyalkenylene linkage, e.g. a C₃ to C₆ alkenylenyl linkage and R is a hydrogen or a unsubstituted or substituted hydrocarbyl group. Preferably, R is a nitrogen-containing hydrocarbyl group, e.g. a poly nitrogen-containing hydrocarbyl group, having 2 or 3 nitrogen atoms, i.e. an amidine or guanidine-containing hydrocarbyl radical. In particular, R may be selected from the group consisting of radicals of the following formulae: R is —N═C(NR₃R₄)(NR₅R₆) or
—NR₇[(NR₃R₄)C═NR₅], i.e. guanidines or —N═C(R₈)(NR₉R₁₀), i.e. amidines, wherein R₃-R₁₀ is H, Alk, Ar or (CH₂)nAr wherein Ar is an aryl group and n is an integer of from 1 to 13 or R₃ and R₄, or R₄ and R₅, or R₅ and R₇, or R₃ and R₇, or R₉ and R₁₀, or R₈ and R₉, together, may be —(CH₂)ₓ—, wherein x is an integer of from 2 to 5, e.g. CH₂—CH₂— or —CH₂—CH₂—CH₂—.

R₂ may be selected from the group consisting of hydroxyl, lower alkyl and hydroxyl-substituted lower alkyl.

For example, R₁ may be —S(CH₂)₂N═C(NH₂)₂ and R₂ may be —CH₂CH(CH₃)₂, —CH₂C(OH)(CH₃)₂, —CH(CH₃)₂ or —CH(CH₃)CH₂CH₃.

### DETAILED DESCRIPTION OF THE INVENTION

The present invention provides a method for enhancing the growth of hair and/or the treatment of alopecia by administering to a patient, preferably by topical application to the scalp of an affected patient, a cyclosporin derivative, i.e. a cyclosporine A derivative, represented by the formula below

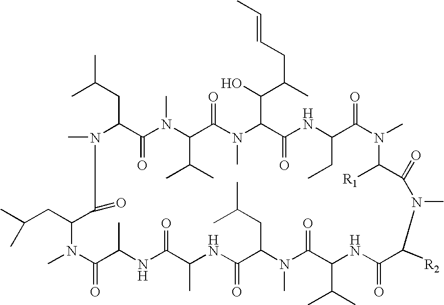

wherein R₁ and R₂ are defined above. In particular R₁ is S-Alk-R wherein Alk is an alkylene linkage, preferably a methylene or poly methylene linkage, e.g. a C₂ to C₆ polymethylene linkage, or a polyalkenylene linkage, e.g. a C₃ to C₆ alkenylenyl linkage and R₂ is selected from the group consisting of hydroxyl, lower alkyl and hydroxyl substituted lower alkyl.

In a first aspect of the invention, R is —N═C(NR₃R₄)(NR₅R₆) or —NR₇[(NR₃R₄)C═NR₅], i.e. a guanidine or —N═C(R₈)(NR₉R₁₀), i.e. an amidine wherein R₃-R₁₀ is H, Alk, Ar or (CH₂)nAr wherein Ar is an aryl group and n is an integer of from 1 to 13 or R₃ and R₄, or R₄ and R₅, or R₅ and R₇, or R₃ and R₇, or R₉ and R₁₀, or R₈ and R₉, together, may be —(CH₂)ₓ—, wherein x is an integer of from 2 to 5, e.g. —CH₂—CH₂— or —CH₂—CH₂—CH₂—.

In a second aspect of the invention, R₁ is a hydrogen atom or a radical of formula (Ia):

—S-Alk-R₁₁ (Ia)

in which
Alk-R₁₁ represents a methyl radical, or alternatively
Alk represents a C₂-C₆ straight chain or branched alkylene radical or a C₃-C₆ cycloalkylene radical, and
R₁₁. represents
a hydrogen atom or a hydroxyl, carboxyl or alkyloxycarbonyl radical, or
an —NR₁₂R₁₃ radical in which R₁₂ and R₁₃, which are identical or different, represent a hydrogen atom or a phenyl, alkyl, C₂-C₄ alkenyl or C₃-C₆ cycloalkyl radical, said radical optionally substituted with selected from a halogen atom, an alkyloxy, alkyloxycarbonyl, amino, alkylamino and dialkylamino radical; or
R₁₂ and R₁₃ represent a benzyl or saturated or unsaturated heterocycylic radical, said heterocycylic radical containing from 5 to 6 ring members and from 1 to 3 heteroatoms;
or in which R₁₂ and R₁₃ form, together with the nitrogen atom to which they are attached, a saturated or unsaturated 4- to 6-membered heterocycle, which heterocycle having an additional heteroatom selected from nitrogen, oxygen and sulphur, and wherein said saturated or unsaturated heterocycle is optionally substituted by an alkyl, phenyl or benzyl radical, or R₁ is a radical of the formula (Ib): —N(R₁₄)—(CH₂)ₙ—NR₁₂R₁₃ in which R₁₂ and R₁₃ are as defined above, R₁₄ represents a hydrogen atom or an alkyl radical and n is an integer ranging from 2 to 4,
and R₂ is selected from the group consisting of hydroxyl, lower alkyl and hydroxyl substituted lower alkyl.
with the proviso that, when R₁ is a hydrogen atom, then R₂ is not an alkyl radical, and wherein the alkyl portions or radicals defined above are straight chain or branched and contain from 1 to 4 carbon atoms, or a pharmaceutically acceptable salt thereof.

In the cyclosporine A derivatives of this second aspect of the invention, the trans butene moiety, which is normally present in the 1-position of cyclosporine A, may be replaced with R₁₅ wherein R₁₅ represents a radical of formula

—CH₂CHCHCH₂—R₁₆ (Ic) or —CH₂SR₁₇ (Id), wherein R₁₆ represents an alkylthio, aminoalkylthio, alkylaminoalkylthio, dialkylaminoalkylthio, pyrimidinylthio, thiazolylthio, N-alkylimidazolylthio, hydroxyalkylphenylthio, hydroxyalkylphenyloxy, nitrophenylamino or 2-oxopyrimidin-1-yl radical and R₁₇ represents an alkyl radical.

This invention also provides pharmaceutical compositions for topical application to enhance hair growth comprising an effective amount of cyclosporine A derivative represented by the above general formula.

### DETAILED DESCRIPTION OF THE PREFERRED EMBODIMENT

Alopecia (baldness) a deficiency of either normal or abnormal hair, is primarily a cosmetic problem in humans. It is a deficiency of terminal hair, the broad diameter, colored hair that is readily seen. However, in the so-called bald person although there is a noticeable absence of terminal hair, the skin does contain vellus hair which is a fine colorless hair which may require microscopic examination to determine its presence. This vellus hair is a precursor to terminal hair. In accordance with the invention as described herein, compounds represented by the general formula, above, can be used to stimulate, such as stimulating the conversion of vellus hair to growth as terminal hair as well as increasing the rate of growth of terminal hair.

For the purpose of describing and claiming the present invention the following terms shall have the following meanings:

“Alkyl” refers to a straight-chain, branched or cyclic saturated aliphatic hydrocarbon. Preferably, the alkyl group has 1 to 12 carbons. More preferably, it is a lower alkyl of from 1 to 7 carbons, most preferably 1 to 4 carbons. Typical alkyl groups include methyl, ethyl, propyl, isopropyl, butyl, isobutyl, tertiary butyl, pentyl, hexyl and the like. The alkyl group may be optionally substituted with one or more substituents are selected from the group consisting of hydroxyl, cyano, alkoxy, ═O, ═S, NO₂, halogen, dimethyl amino and SH.
“Alkenyl” refers to a straight-chain, branched or cyclic unsaturated hydrocarbon group containing at least one carbon—carbon double bond. Preferably, the alkenyl group has 2 to 12 carbons. More preferably it is a lower alkenyl of from 2 to 7 carbons, most preferably 2 to 4 carbons. The alkenyl group may be optionally substituted with one or more substituents selected from the group consisting of hydroxyl, cyano, alkoxy, O, S, NO₂, halogen, dimethyl amino and SH.
“Aryl” refers to an aromatic group which has at least one ring having a conjugated pi electron system and includes carbocyclic aryl, heterocyclic aryl and biaryl groups. The aryl group may be optionally substituted with one or more substituents selected from the group consisting of halogen, trihalomethyl, hydroxyl, SH, OH, NO₂, amine, thioether, cyano, alkoxy, alkyl, and amino.
“Alkaryl” refers to an alkyl that is covalently joined to an aryl group. Preferably, the alkyl is a lower alkyl.
“Alkoxy” refers to an “O-alkyl” group.
“bOC” refers to a t-butyloxycarbonyl protecting group.
“Carbocyclic aryl” refers to an aryl group wherein the ring atoms are carbon.
“Heterocyclic aryl” refers to an aryl group having from 1 to 3 heteroatoms as ring atoms, the remainder of the ring atoms being carbon. Heteroatoms include oxygen, sulfur, and nitrogen.
“Hydrocarbyl” refers to a hydrocarbon radical having only carbon and hydrogen atoms. Preferably, the hydrocarbyl radical has from 1 to 20 carbon atoms, more preferably from 1 to 12 carbon atoms and most preferably from 1 to 7 carbon atoms.
“Substituted hydrocarbyl” refers to a hydrocarbyl radical wherein one or more, but not all, of the hydrogen and/or the carbon atoms are replaced by a halogen, nitrogen, oxygen, sulfur or phosphorus atom or a radical including a halogen, nitrogen, oxygen, sulfur or phosphorus atom, e.g. fluoro, chloro, cyano, nitro, hydroxyl, phosphate, thiol, etc.
One class of cyclosporine A derivatives useful in the method of this invention are prepared as follows:
Compounds where R₄, R₅ and R₆ are Hydrogen and R₇ is hydrogen, alkyl, substituted alkyl or aryl may be prepared by reaction of a compound of formula (I) where X is a leaving group and P is a protecting group with a compound of formula (II) in a suitable solvent such as methanol to afford compounds of formula (III). For compounds of formula I, typical examples of the protecting group are where X=chlorine, MeS, MeSO2, 1-imidazolyl and especially 1-pyrazolyl. Protecting groups P are preferably tertiary butyloxycarbonyl groups (tBoc) groups.

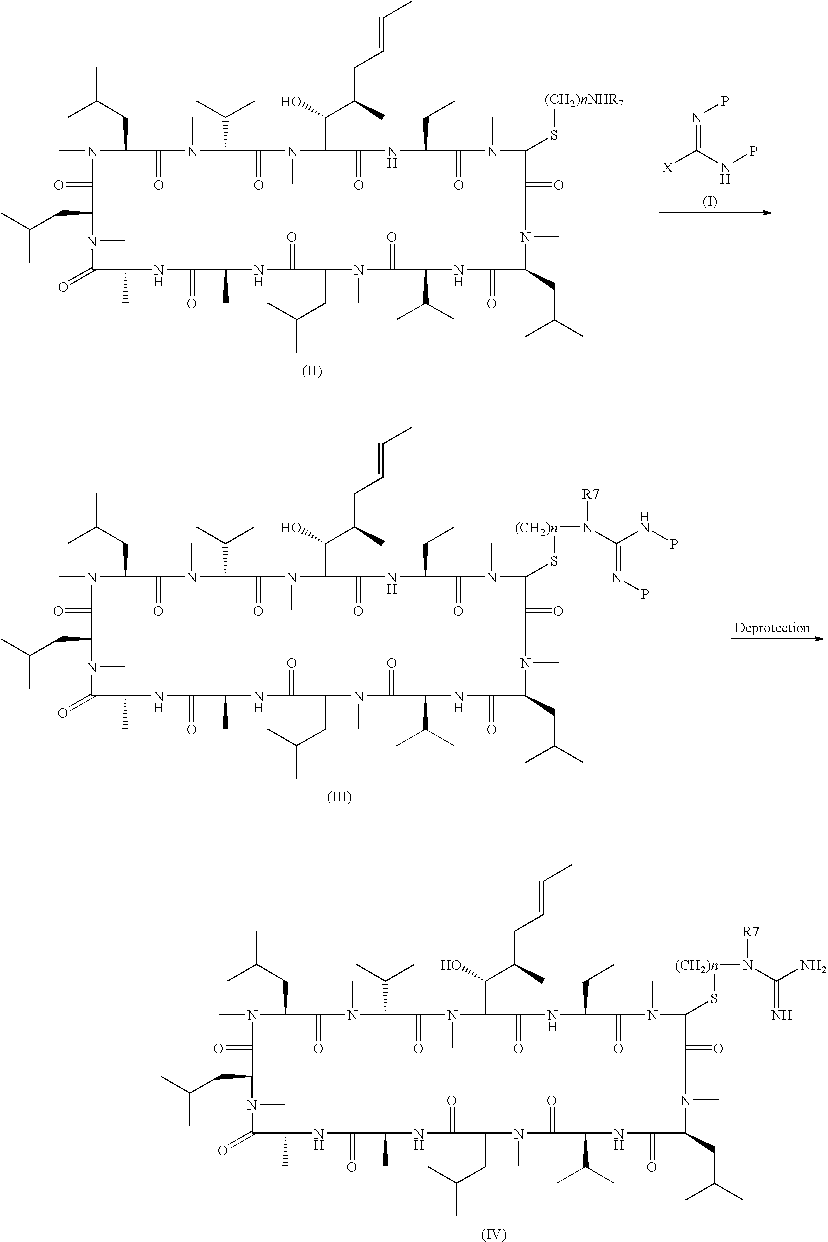

Compounds of formula (III) may be de-protected under a variety of conditions to provide compounds of formula (IV). For example, when P=tertiary butyloxycarbonyl groups (tBoc), this may be removed under acidic conditions using acids such as methanesulphonic acid.

Compounds of formula (V) where R₇ is hydrogen, alkyl, substituted alkyl or aryl; R₃ is alkyl, substituted alkyl or aryl, may be prepared by reaction of a compound of formula (VI) where X is a leaving group and P is a protecting group with a compound of formula (II) in a suitable solvent such as methanol to afford compounds of formula (VII).

For compounds of formula (VI), typical examples of the protecting group are where X=chlorine, MeS, MeSO2, 1-imidazolyl and especially 1-pyrazolyl. Protecting groups P are preferably tertiary butyloxycarbonyl groups (tBoc) groups.

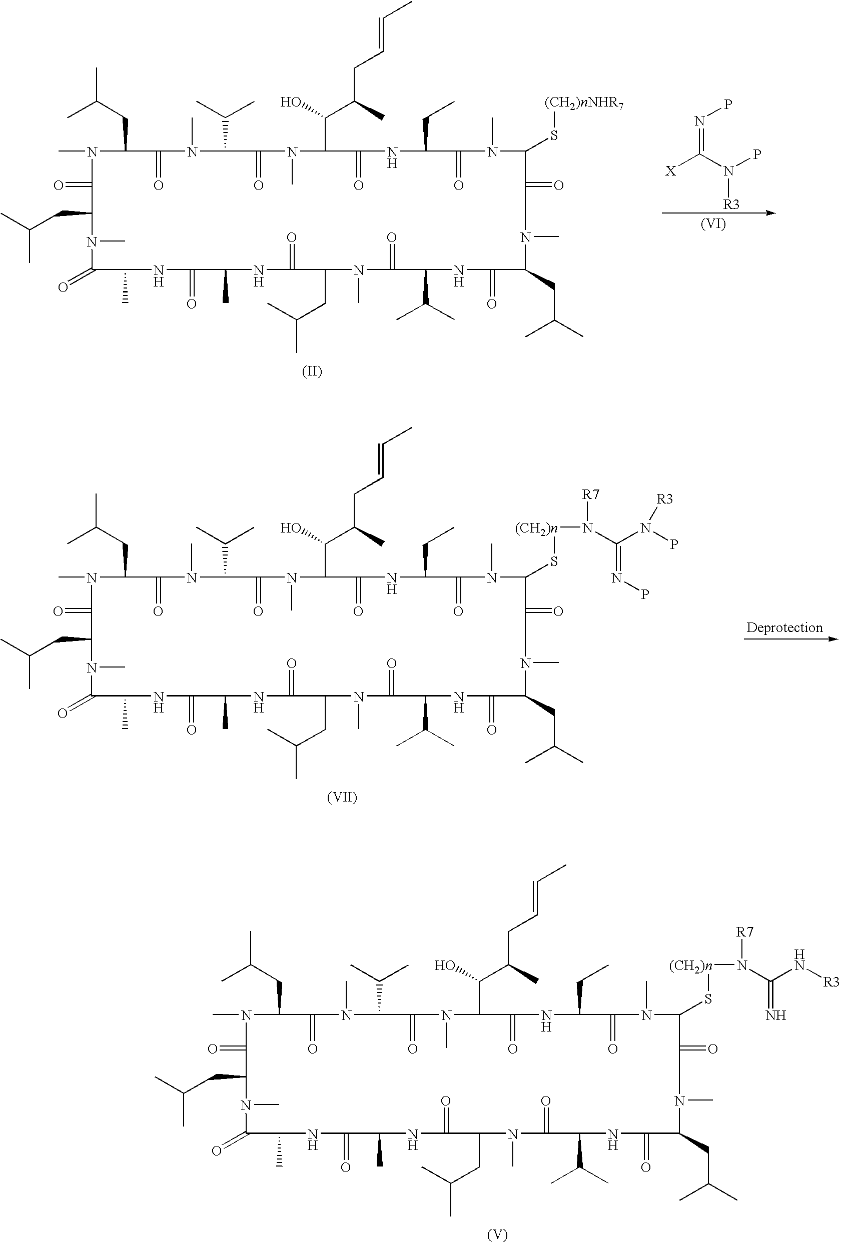

For example, in WO/2003/051797 N,N′-Di-tBoc-N-methyl-1H-pyrazole-1-carboxamidine has been used to prepare an N-methyl guanidine in an unrelated chemical family.

Other compounds of the invention may be made in similar ways using related synthetic methods with, if appropriate, suitable protecting groups compatible with the synthetic methodology.

Compounds of formula (X) where R═—N═C(R₈)—NR₉R₁₀ (amidines) where R₈ is hydrogen alkyl, substituted alkyl or aryl and R₉ and R₁₀ can be alkyl, substituted alkyl or aryl or R₉ and R₁₀ can form a ring may be prepared by reaction of a compound of formula (VIII) with a compound of formula (IX) to afford compounds of formula (X).

R₁₁ is preferably lower alkyl and typical examples of compound (VIII) are Dimethylformamide dimethylacetal (DMF.DMA) and Dimethylacetamide diemthylacetal (DMA.DMA).

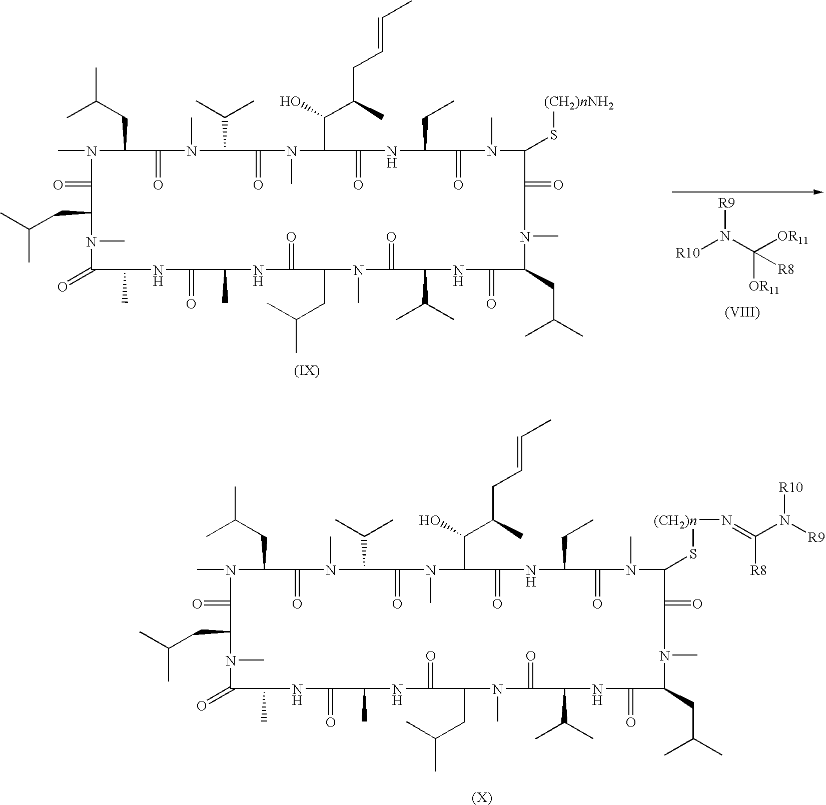

Below are specific examples of the preparation of certain cyclosporine A derivatives useful in the method of enhancing hair growth of the present invention by the above general procedures.

### Guanidine and Amidine Analogues of 3-[(2-aminoethylthio]-cyclosporin A

### Example A

### 3-[(2-Guanidyl)-ethylthio]-cyclosporin A (III)

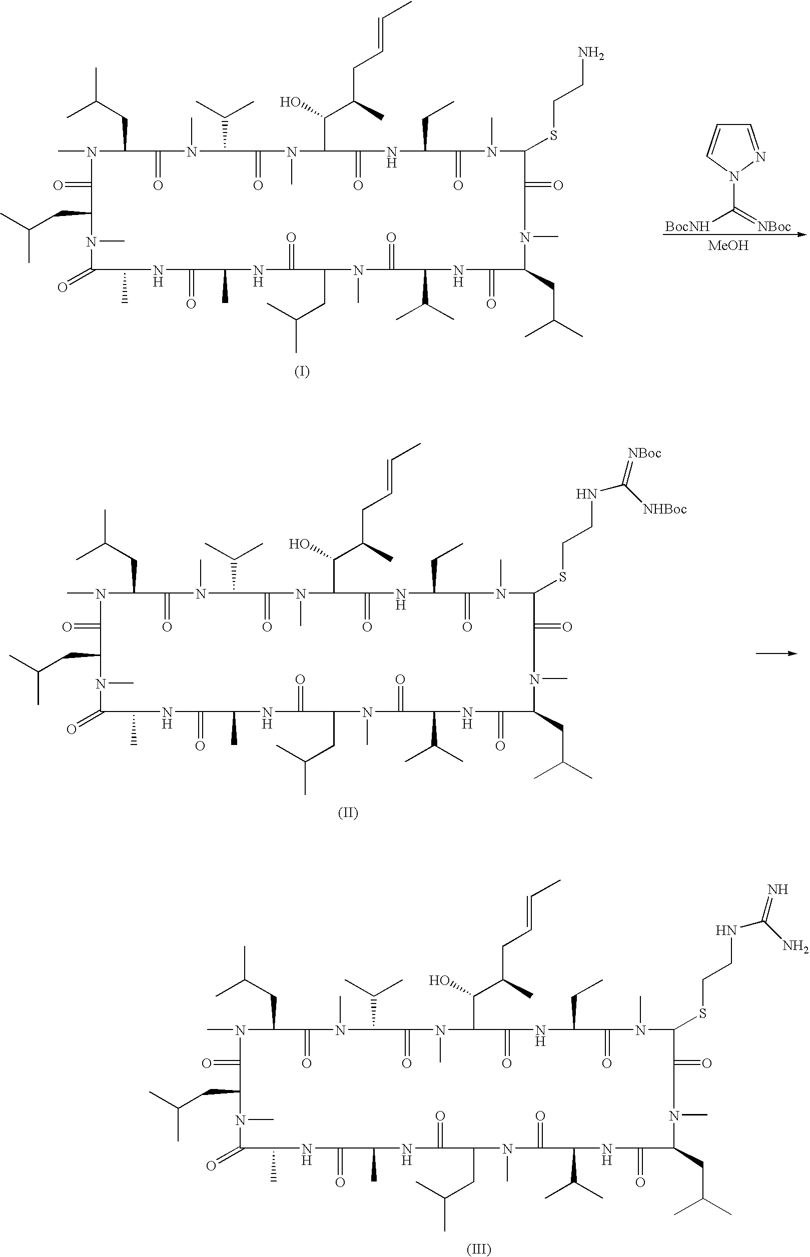

To a solution of 3-[(2-aminoethylthio]-cyclosporin A*-(I)) (200 mg, 0.16 mmol) in methanol (20 mL) was added di-Boc-pyrazole carboxamidine (250 mg, 0.8 mmol), and the reagents were stirred together for 18 h. After this time, a further portion of the di-Boc-pyrazole carboxamidine (100 mg, 0.32 mmol) was added and the reaction was stirred for a further 3 h. The reaction was then reduced in vacuo, redissolved in dichloromethane, washed with 0.5M citric acid, and the organic layer was dried over MgSO₄ and reduced in vacuo. The product was then purified by chromatography column on a 10 g SPE cartridge eluting with diethyl ether to isolate 90 mg (40%) of desired product (II).

As the first member of the Guanidine and Amidine examples synthesized and because of the difficulties anticipated in characterising the final product (III), it was decided to fully and extensively characterize the di-Boc protected guanidine (II) at this stage and to then take this material onto the free guanidine (III) by acid hydrolysis. Subsequent analogues in this Guanidine and Amidine subclass made from 3-[(2-aminoethylthio]-cyclosporin A were then characterised principally by MS

Compound (II) was analysed by ¹H, ¹³C, DEPT NMR and subsequently by a series of 2-D NMR experiments, HMQC, HMBC and DEPT-HMQC.

Presence of the 3-[(2-Guanidyl)-ethylthio] side chain was confirmed by 1D & 2D NMR. Analysis was performed in CDCl₃ solution at 300K on a Bruker DRX500 spectrometer.

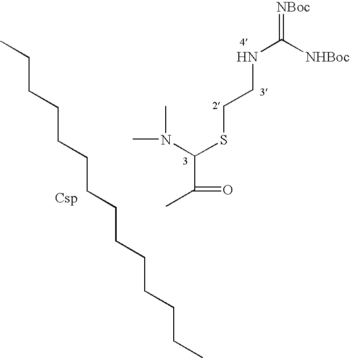

¹H NMR Key Resonances:

δ=1.50, 1.51 ppm (2 singlets, 2×Boc, 18H, 6×CH3.)

δ=5.89 ppm, (singlet, sarcosine, 1H).

### 2D Spectra

Using ¹H detected Hetronuclear Multiple Quantum Coherence (HMQC), Hetronuclear Multiple Bond Correlation (HMBC) and edited Hetronuclear Single Quantum Coherence (DEPT-HSQC) experiments, connectivity and assignment may be made confirming the presence of the 3-[(2-Guanidyl)-ethylthio] side chain.

H (3) to 2′ (multiplet, ¹H 2.84 ppm, 2H).
2′ to 3′ (multiplet, ¹H 3.67 ppm, 2H).
3′ to NH 4′ (triplet J_{HH} 5.8 Hz, ¹H 8.67 ppm, 1H).

To a solution of the di-Boc protected 3-[(2-Guanidyl)-ethylthio]-cyclosporin A (II) (21 mg, 0.0138 mmol) in dichloromethane (0.3 mL) was added trifluoroacetic acid (0.3 mL) and the solution was stirred at room temperature for 1 hour. The solution was concentrated to give the product (III) as a white solid (20 mg; 100%)

Analysis by MS (E⁺) showed a mass of 1320.2 (M+H) consistent with the proposed structure

### Example B

### 3-[(2-N,N-dimethylformamidinyl)-1-thioethyl]-cyclosporin A (III)

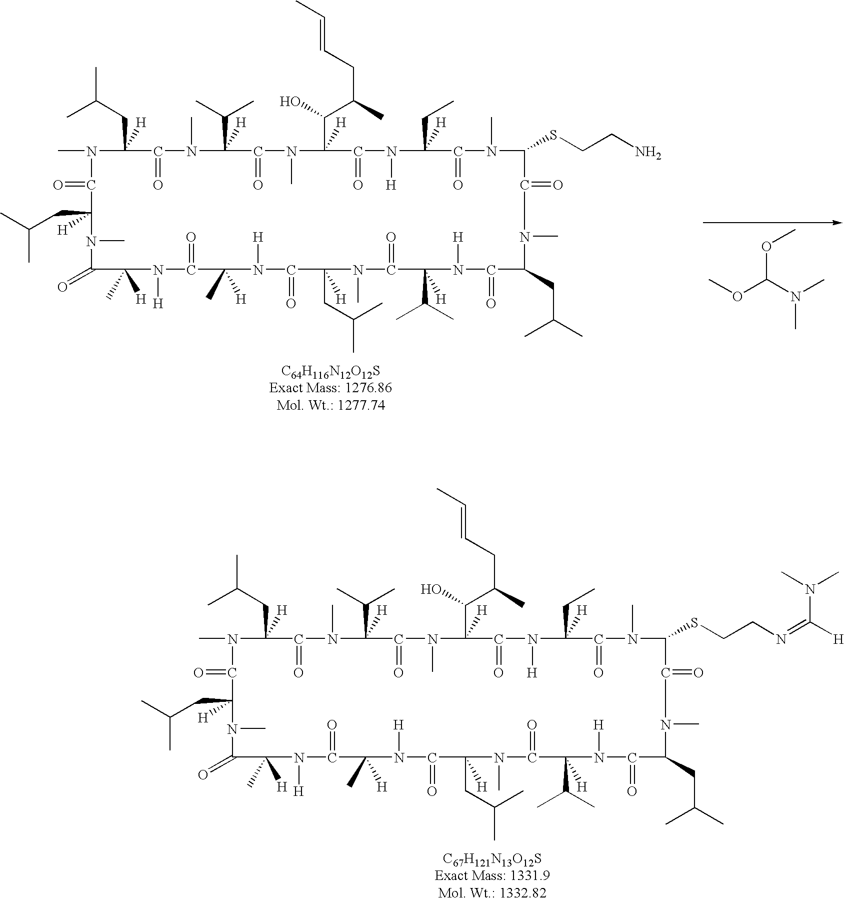

A mixed solution of 3-(1-thioethylamine)cyclosporine A (0.64 g, 0.5 mmol) and N,N-dimethylformamide dimethyl acetal in 20 mL of THF was refluxed for two hours. After removal of solvent under vacuum, the residue was subject to silica gel column using methylene/methanol (10:1) as eluents, 300 mg of pure product was obtained (yield: 45.0%)

MS (E+) showed a mass of 1332.82 (M+H⁺) consistent with the proposed structure.

Other methods will be apparent to a chemist skilled in the art as will methods for preparing starting materials and intermediates etc

In accordance with the present invention, the cyclosporin A derivatives may be applied to an affected patient in any efficacious concentration, e.g., 0.01 to saturation (e.g. greater than 20 weight percent) in a pharmaceutically acceptable excipient. From 0.01 to 50 weight percent, preferably from 0.1 to 20 weight percent, of the cyclosporin A derivative in a pharmaceutically acceptable excipient may be used. Such pharmaceutically acceptable excipients are, for example, animal oil, vegetable oil, an appropriate organic or aqueous solvent, a natural or synthetic polymer, or an appropriate membrane to encapsulate the cyclosporin A derivative.

The invention is also related to dermatological compositions for topical treatment for the stimulation of hair growth which comprise an effective hair growth stimulating amount of one or more compounds as defined above and a dermatologically compatible carrier. Effective amounts of the active compounds may be determined by one of ordinary skill in the art but will vary depending on the compound employed, frequency of application and desired result, and the compound will generally range from about 0.0000001 to about 50%, by weight, of the dermatological composition, preferably from about 0.001 to about 50%, by weight, of total dermatological composition, more preferably from about 0.1 to about 30%, by weight of the composition.

The present invention finds application in all mammalian species, including both humans and animals. In humans, the compounds of the subject invention can be applied for example, to the scalp, face, beard, head, pubic area, upper lip, eyebrows, and eyelids. In animals raised for their pelts, e.g., mink, the compounds can be applied over the entire surface of the body to improve the overall pelt for commercial reasons. The process can also be used for cosmetic reasons in animals, e.g., applied to the skin of dogs and cats having bald patches due to mange or other diseases causing a degree of alopecia.

The pharmaceutical compositions contemplated by this invention include pharmaceutical compositions suited for topical and local action.

The term “topical” as employed herein relates to the use of a compound, i.e. a cyclosporine A derivative, as described herein, incorporated in a suitable pharmaceutical carrier, and applied at the site of thinning hair or baldness for exertion of local action. Accordingly, such topical compositions include those pharmaceutical forms in which the compound is applied externally by direct contact with the skin surface to be treated. Conventional pharmaceutical forms for this purpose include ointments, liniments, creams, shampoos, lotions, pastes, jellies, sprays, aerosols, and the like, and may be applied in patches or impregnated dressings depending on the part of the body to be treated. The term “ointment” embraces formulations (including creams) having oleaginous, water-soluble and emulsion-type bases, e.g., petrolatum, lanolin, polyethylene glycols, as well as mixtures of these.

Typically, the compounds are applied repeatedly for a sustained period of time topically on the part of the body to be treated, for example, the eyelids, eyebrows, skin or scalp. The preferred dosage regimen will generally involve regular, such as daily, administration for a period of treatment of at least one month, more preferably at least three months, and most preferably at least six months.

For topical use on the eyelids or eyebrows, the active compounds can be formulated in aqueous alcohol solutions, creams, ointments or oils exhibiting physiologically acceptable osmolarity by addition of pharmacologically acceptable buffers and salts. Such formulations may or may not, depending on the dispenser, contain preservatives such as benzalkonium chloride, chlorhexidine, chlorobutanol, parahydroxybenzoic acids and phenylmercuric salts such as nitrate, chloride, acetate, and borate, or antioxidants, as well as additives like EDTA, sorbitol, boric acid etc. as additives. Furthermore, particularly aqueous solutions may contain viscosity increasing agents such as polysaccharides, e.g., methylcellulose, mucopolysaccharides, e.g., hyaluronic acid and chondroitin sulfate, or polyalcohol, e.g., polyvinylalcohol. Various slow releasing gels and matrices may also be employed as well as soluble and insoluble ocular inserts, for instance, based on substances forming in-situ gels. Depending on the actual formulation and compound to be used, various amounts of the drug and different dose regimens may be employed. Typically, the daily amount of compound for treatment of the eyelid may be about 0.1 ng to about 100 mg per eyelid.

For topical use on the skin and the scalp, the compound can be advantageously formulated using ointments, creams, liniments or patches as a carrier of the active ingredient. Also, these formulations may or may not contain preservatives, depending on the dispenser and nature of use. Such preservatives include those mentioned above, and methyl-, propyl-, or butyl-parahydroxybenzoic acid, betain, chlorhexidine, benzalkonium chloride, and the like. Various matrices for slow release delivery may also be used. Typically, the dose to be applied on the scalp is in the range of about 0.1 ng to about 100 mg per day, more preferably about 1 ng to about 10 mg per day, and most preferably about 10 ng to about 1 mg per day depending on the compound and the formulation. To achieve the daily amount of medication depending on the formulation, the compound may be administered once or several times daily with or without antioxidants.

The invention is further illustrated by the following non-limiting examples:

### Example 1

### In Vivo Treatment

A study is initiated to systematically evaluate the appearance of lashes and hair around the eyes of patients who are administering 3-[(2-Guanidyl)-ethylthio]-cyclosporin A (the cyclosporine A derivative of Example A, above) in only one eye. The study involves 10 subjects, 5 male, 5 female, average age 70 years, (ranging from 50-94 years). All patients have glaucoma. Each subject is treated daily by the topical application of one drop of the composition of Example 3, below, to provide 3-[(2-Guanidyl)-ethylthio]-cyclosporin A at a dosage of 1.5 .mu.g/ml/eye/day, to the region of one eye by instilling the drop onto the surface of the eye. The region of the fellow control eye is not treated with 3-[(2-Guanidyl)-ethylthio]-cyclosporin A and serves as a control. The mean duration of exposure to 3-[(2-Guanidyl)-ethylthio]-cyclosporin A prior to assessing the parameter of lash growth between the control and study eye is 129 days (range 90-254 days). Observations are made under high magnification at the slit lamp biomicroscope. Documentation of differences between the control and treatment areas is accomplished using a camera specially adapted for use with the slit lamp biomicroscope.

The results of the observations are as follows:
Length of lashes: Increased length of eyelashes is regularly observed on the side treated with 3-[(2-Guanidyl)-ethylthio]-cyclosporin A.
Number of lashes: Increased numbers of lashes are observed in the treated eye of each patient. In areas where there are a large number of lashes in the control eye, the increased number of lashes in the 3-[(2-Guanidyl)-ethylthio]-cyclosporin A-treated eye gave the lashes on the treated side a more thickly matted overall appearance.
Increased growth of vellus hair on lids: Fine microscopic vellus hair is present on the skin of the lids and is easily seen with the slit lamp biomicroscope. This vellus hair is typically denser adjacent to and below the lateral portion of the lower lids. While remaining microscopic, vellus hairs are increased in number, appear more robust and are much longer and thicker in treated than in control eyes in the areas below and lateral to the lower lid.
Perpendicular angulation of hairs: In areas where there are lash-like hairs above the lash line and in the medial and lateral canthal areas, the hairs are much longer, thicker and heavier. They also leave the surface of the skin at a more acute angle, as though they are stiffer or held in a more erect position by more robust follicles. This greater incline, pitch, rise or perpendicular angulation from the skin surface gives the appearance of greater density of the hairs.

The foregoing observations establish that 3-[(2-Guanidyl)-ethylthio]-cyclosporin A can be used to increase the growth of hair in man.

### Example 2

### Topical Composition

A topical composition is prepared as follows: Individual oil-phase and water-phase ingredients were mixed in a separate container, and each mixture was completely dissolved by heating to 80.degree. C. Two phases of the ingredients were mixed, emulsified, and cooled to room temperature to prepare the hair cream, with the composition as shown in Table 1, below. Water was added to adjust to 100% the total weight including the oil-phase and water-phase ingredients.

It is found that the composition of Table 1 has a hair growth promoting effect at a level similar to a conventional hair cream containing 0.1% cyclosporin A, as evaluated in an animal experiment according to the Test Example of Column 22 of U.S. Pat. No. 6,987,090, the disclosure of which is incorporated by reference.

TABLE 1

| | |
|---|---|
| Paraffin | 5.0 |
| Cetosteryl alcohol | 5.5 |
| Petrolatum | 5.5 |
| Glycerin monostearate | 3.0 |
| Polyoxyethylenedodecylether | 3.0 |
| Propylparaben | 0.3 |
| 3-[(2-Guanidyl)-ethylthio]-cyclosporin A | 0.1 |
| Glycerin | 7.0 |
| Polyethylene glycol | 20.0 |
| Water | Balance |

The composition is applied to bald human scalp once daily to stimulate the growth of hair.

### Example 3

### Shampoo and Hair Conditioner Formulations

A topical formulation that can be used as a shampoo or hair conditioner formulation can be prepared according to the teachings of U.S. Pat. No. 6,987,090 by substituting 3-[(2-Guanidyl)-ethylthio]-cyclosporin A for the cyclosporine A derivative disclosed therein.

### Example 4

### Topical Ointment

An ointment containing 2% by weight 3-[(2-Guanidyl)-ethylthio]-cyclosporin A is prepared as follows:

White petrolatum is melted, strained and liquid petrolatum is added thereto. The 3-[(2-Guanidyl)-ethylthio]-cyclosporin A, zinc oxide, and calamine are added to liquid petrolatum and the mixture milled until finely divided and uniformly dispersed. The mixture is stirred into the white petrolatum, melted and cooled with stirring until the ointment congeals.

The foregoing ointment can be applied topically to mammalian skin for increased rate of hair growth, and can be prepared by omitting the zinc oxide and calamine.

### Example 5

### Ointment

A dermatological ophthalmic ointment containing 10% by weight 3-[(2-Guanidyl)-ethylthio]-cyclosporin A is prepared by adding the active compound to light liquid petrolatum. White petrolatum is melted, strained, and the temperature adjusted to 45-50° C. Liquid petrolatum slurry is added and the ointment stirred until congealed. Suitably the ointment is packaged in 30 gm tubes.

The foregoing ointment can be applied to the eyelid to enhance the growth of eyelashes. Similarly the composition can be applied to the brow for eyebrow growth.

### Example 6

### Tonic

A solution containing 0.5%, by weight, 3-[(2-Guanidyl)-ethylthio]-cyclosporin A is prepared as follows. 3-[(2-Guanidyl)-ethylthio]-cyclosporin A is dissolved in a suitable alcohol, e.g. ethanol, and to the resulting solution is added tocopherol acetate, salicylic acid, L-menthol and Tween 20 to provide the hair tonic of Table 2, below. The solution is aseptically filled into sterile containers.

TABLE 2

| | |
|---|---|
| Ethanol | 40.0 |
| 3-[(2-Guanidyl)-ethylthio]-cyclosporin A | 0.5 |
| Tocopherol | 0.1 |
| Salicylic Acid | 0.1 |
| L-menthol | 0.3 |
| Tween 20 | 0.5 |
| Water | Balance |

The composition so prepared can be used in the topical treatment of baldness by application to the scalp daily.

### Example 7

### Lotion

3-[(2-Guanidyl)-ethylthio]-cyclosporin A is dissolved in a vehicle of N-methyl pyrrolidone and propylene glycol. The composition can be used for application to dogs or cats having hair loss due to mange or alopecia of other causes.

### Example 8

### Aerosol

An aerosol containing approximately 0.1% by weight 3-[(2-Guanidyl)-ethylthio]-cyclosporin A is prepared by dissolving the 3-[(2-Guanidyl)-ethylthio]-cyclosporin A in absolute alcohol. The resulting solution filtered to remove particles and lint. This solution is chilled to about minus 30° C. To the solution is added a chilled mixture of dichlorodifluoromethane and dichlorotetrafluoroethane.

Thirteen ml plastic-coated amber bottles are cold filled with 11.5 gm each of the resulting solution and capped.

The composition can be sprayed on the scalp daily to stimulate the growth of hair.

### Example 9

### Dusting Powder

A powder of the compound 3-[(2-Guanidyl)-ethylthio]-cyclosporin A is prepared by mixing in dry form with talcum powder at a weight/weight ratio of 1:10. The powdered mixture is dusted on the fur of minks or other commercially valuable fur bearing animals and show animals for increased rate of hair growth.

### Example 10

### Related Compounds

Following the procedure of the preceding Examples, compositions are similarly prepared substituting an equimolar amount of the amidine compound of Example B for the 3-[(2-Guanidyl)-ethylthio]-cyclosporin A disclosed in the preceding Examples. Similar results are obtained.

While the preferred embodiment of the invention has been illustrated and described, it will be appreciated that various changes can be made therein without departing from the spirit and scope of the invention.

The embodiments of the invention in which an exclusive property or privilege is claimed are defined as follows:

Specific examples of these pharmaceutically acceptable excipients are olive oil, arachis oil, castor oil, mineral oil, petroleum jelly, dimethyl sulphoxide, chremophor, Miglyol 182 (commercially available from Dynamit Nobel Kay-Fries Chemical Company, Mont Vale, N.J.), an alcohol (e.g. ethanol, n-propyl alcohol, or iso-propyl alcohol), liposomes or liposome-like products or a silicone fluid. Preferred excipients are dimethyl sulphoxide and olive oil. Mixtures of at least two of any suitable excipients may be used.

An example of a useful polymeric excipient is a polyoxyethylated castor oil.

Examples of pharmaceutically acceptable membranes which can be advantageously used in the practice of this invention are microdone, an artificial lipid membrane, polyvinyl alcohol, or methylcellulose.

The cyclosporin A derivatives are advantageously administered topically as a solution, suspension, or ointment containing an effective amount of the derivative. Concentrations of 0.01 to 50 weight percent, preferably 0.1 to 20 weight percent, of the cyclosporin A derivatives may be used in the practice of the present invention.

In accordance with a method of the present invention, at least one of the cyclosporin A derivatives is administered topically in any quantity required to provide the degree of treatment needed. For example, 5 microliters to 1 milliliter of a solution, suspension, or ointment containing an effective amount of the cyclosporin A derivative, such as 0.01 to 50 weight percent, preferably 0.1 to 20 weight percent, of the cyclosporin A derivative is advantageously used.

Further objects of this invention, together with additional features contributing thereto and advantages accruing therefrom will be apparent from the following examples of the invention.

The foregoing description details specific methods and compositions that can be employed to practice the present invention, and represents the best mode contemplated. Thus, however detailed the foregoing may appear in text, it should not be construed as limiting the overall scope hereof; rather, the ambit of the present invention was to be governed only by the lawful construction of the appended claims. In particular, although the method of the present invention has been described with the use of the specific cyclosporine A derivatives of the above formula, the novel cyclosporine derivatives that may be used in the method of the present invention further include 3-substituted iminoalkylthio cyclosporin A derivatives, preferably 3-substituted diaminoiminoalkylthio cyclosporin A derivatives, e.g. ((R)-(diamino)iminoalkyllthio-Sar)³-(4′-hydroxy-MeLeu)⁴ cyclosporin A, ((R)-(alkyl)(dialkylamino)iminoalkylthio-Sar)³-(4′-hydroxy-MeLeu)⁴-cyclosporin A, ((R)-(alkyl)(dialkylamino)iminoalkylthio-Sar)³-cyclosporin A derivatives and ((R)-(diamino)iminoalkylthio-Sar)³-cyclosporin A derivatives.

What is claimed is:

1. A method for the treatment of alopecia in a patient, said method comprising administering, topically to the scalp of the patient, a therapeutically effective amount of a cyclosporine A derivative represented by the following formula: 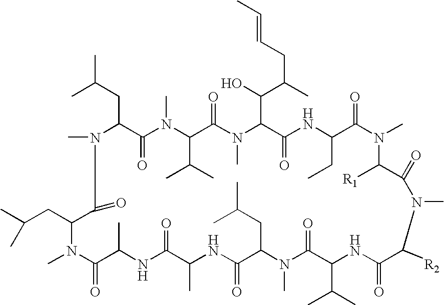
wherein R₁ is S-Alk-R wherein Alk is an alkylene or alkylenyl linkage, R is a hydrogen or a unsubstituted or substituted hydrocarbyl group and R₂ is selected from the group consisting of hydroxyl, lower alkyl and hydroxyl substituted lower alkyl.

2. The method of claim 1 wherein R₁ is a methylene or a C₂ to C₆ polymethylene linkage.

3. The method of claim 1 wherein R₁ is a C₃ to C₆ alkenylenyl linkage.

4. The method of claim 1 wherein R is —N═C(NR₃R₄)(NR₅R₆) or —NR₇C(NR₃)(C═NR₅), wherein R₃-R₇ is H, Alk, Ar or (CH₂)nAr wherein Ar is an aryl group and n is an integer of from 1 to 13 or R₃ and R₄ or R₄ and R₅ or R₅ and R₇ or R₃ and R₇, together may be —CH₂—CH₂— or —CH₂—CH₂—CH₂—.

5. The method of claim 1 wherein said cyclosporine A derivative is a 3-substituted diaminoiminoalkylthio cyclosporine A derivative.

6. The method of claim 1 wherein said cyclosporine A derivative is selected from the group consisting of ((R)-(diamino)iminoalkyllthio-Sar)³-(4′-hydroxy-MeLeu)⁴ cyclosporin A derivatives, ((R)-(alkyl)(dialkylamino)iminoalkylthio-Sar)³-(4′-hydroxy-MeLeu)⁴-cyclosporin A, ((R)-(alkyl)(dialkylamino)iminoalkylthio-Sar)³-cyclosporin A derivatives and ((R)-(diamino)iminoalkylthio-Sar)³-cyclosporin A derivatives

7. The method of claim 1 wherein said cyclosporine A derivative is selected from the group of compounds according to claim 1 wherein R₁ is —S(CH₂)₂N═C(NH₂)₂ and R₂ is —CH₂CH(CH₃)₂, —CH₂C(OH)(CH₃)₂, —CH(CH₃)₂ or —CH(CH₃)CH₂CH₃.

8. A method for stimulating hair growth in a mammalian species comprising applying to mammalian skin an effective amount of a cyclosporine derivative represented by the following formula: 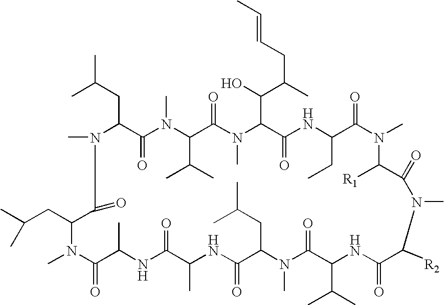
wherein R₁ is S-Alk-R wherein Alk is an alkylene or alkylenyl linkage, R is a hydrogen or a unsubstituted or substituted hydrocarbyl group and R₂ is selected from the group consisting of hydroxy and lower alkyl.

9. The method of claim 8 wherein R₁ is a methylene or a C₂ to C₆ polymethylene linkage.

10. The method of claim 8 wherein R₁ is a C₃ to C₆ alkenylenyl linkage.

11. The method of claim 8 wherein R is —N═C(NR₃R₄)(NR₅R₆) or —NR₇C(NR₃)(C═NR₅), wherein R₃-R₇ is H, Alk, Ar or (CH₂)nAr wherein Ar is an aryl group and n is an integer of from 1 to 13 or R₃ and R₄ or R₄ and R₅ or R₅ and R₇ or R₃ and R₇, together may be —CH₂—CH₂— or —CH₂—CH₂—CH₂—.

12. The method of claim 8 wherein said cyclosporine A derivative is a 3-substituted diaminoiminoalkylthio cyclosporine A derivative.

13. The method of claim 8 wherein said cyclosporine A derivative is selected from the group consisting of ((R)-(diamino)iminoalkyllthio-Sar)³-(4′-hydroxy-MeLeu)⁴ cyclosporin A derivatives, ((R)-(alkyl)(dialkylamino)iminoalkylthio-Sar)³-(4′-hydroxy-MeLeu)⁴-cyclosporin A, ((R)-(alkyl)(dialkylamino)iminoalkylthio-Sar)³-cyclosporin A derivatives and ((R)-(diamino)iminoalkylthio-Sar)³-cyclosporin A derivatives

14. The method of claim 8 wherein said cyclosporine A derivative is selected from the group of compounds according to claim 1 wherein R₁ is —S(CH₂)₂N═C(NH₂)₂ and R₂ is —CH₂CH(CH₃)₂, —CH₂C(OH)(CH₃)₂, —CH(CH₃)₂ or —CH(CH₃)CH₂CH₃

15. A method for converting vellus hair or intermediate hair to terminal hair comprising applying to mammalian skin at the locale of vellus hair an effective amount of a cyclosporine compound represented by the following formula: 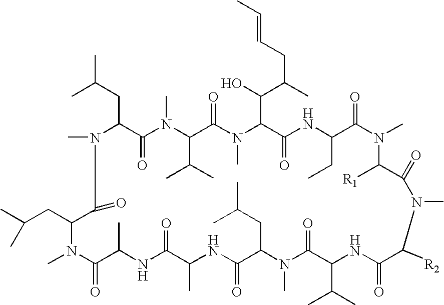
wherein R₁ is S-Alk-R wherein Alk is an alkylene or alkylenyl linkage, R is a hydrogen or a unsubstituted or substituted hydrocarbyl group and R₂ is selected from the group consisting of hydroxy and lower alkyl.

16. A method of increasing one or more of length, thickness, number, and density, of eyelash hair or eyebrow hair, comprising administering an effective amount of a compound of the following formula: 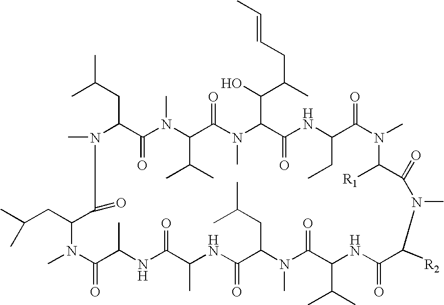
wherein R₁ is S-Alk-R wherein Alk is an alkylene or alkylenyl linkage, R is a hydrogen or a unsubstituted or substituted hydrocarbyl group and R₂ is selected from the group consisting of hydroxyl and lower alkyl to a person on the area where hair growth is desired.

17. The method of claim 16 wherein the effective amount of said compound is administered in the form of a liquid composition containing about 0.03% by weight of said compound is administered to the person.

18. The method of claim 17 wherein said compound is administered to an eyelid margin.

19. The method of claim 17 wherein said compound is administered to an eyebrow.

20. A method for stimulating hair growth in a mammalian species comprising the application to mammalian skin of an effective amount of a cyclosporine derivative represented by the following formula: 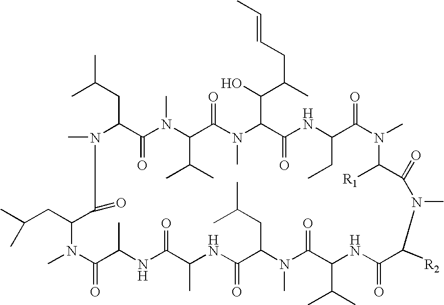
R₁ is a hydrogen atom or a radical of formula (Ia):
—S-Alk-R₁₁ (Ia)
in which
Alk-R₁₁ represents a methyl radical, or alternatively
Alk represents a C₂-C₆ straight chain or branched alkylene radical or a C₃-C₆ cycloalkylene radical, and
R₁₁. represents
a hydrogen atom or a hydroxyl, carboxyl or alkyloxycarbonyl radical, or
an —NR₁₂R₁₃ radical in which R₁₂ and R₁₃, which are identical or different, represent a hydrogen atom or a phenyl, alkyl, C₂-C₄ alkenyl or C₃-C₆ cycloalkyl radical, said radical optionally substituted with selected from a halogen atom, an alkyloxy, alkyloxycarbonyl, amino, alkylamino and dialkylamino radical; or
R₁₂ and R₁₃ represent a benzyl or saturated or unsaturated heterocycylic radical, said heterocycylic radical containing from 5 to 6 ring members and from 1 to 3 heteroatoms;
or in which R₁₂ and R₁₃ form, together with the nitrogen atom to which they are attached, a saturated or unsaturated 4- to 6-membered heterocycle, which heterocycle having an additional heteroatom selected from nitrogen, oxygen and sulphur, and wherein said saturated or unsaturated heterocycle is optionally substituted by an alkyl, phenyl or benzyl radical, or R₁ is a radical of the formula (Ib): —N(R₁₄)—(CH₂)ₙ—NR₁₂R₁₃ in which R₁₂ and R₁₃ are as defined above, R₁₄ represents a hydrogen atom or an alkyl radical and n is an integer ranging from 2 to 4,
and R₂ is selected from the group consisting of hydroxyl, lower alkyl and hydroxyl substituted lower alkyl,
with the proviso that, when R₁ is a hydrogen atom, then R₂ is not an alkyl radical, and wherein the alkyl portions or radicals defined above are straight chain or branched and contain from 1 to 4 carbon atoms, or a pharmaceutically acceptable salt thereof.

21. The method of claim 20 wherein the trans butene moiety at the 1-position of cyclosporine A is replaced with R₁₅, wherein R₁₅ represents a radical of formula
—CH₂CHCHCH₂—R₁₆ (Ic) or —CH₂SR₁₇ (Id), wherein R₁₆ represents an alkylthio, aminoalkylthio, alkylaminoalkylthio, dialkylaminoalkylthio, pyrimidinylthio, thiazolylthio, N-alkylimidazolylthio, hydroxyalkylphenylthio, hydroxyalkylphenyloxy, nitrophenylamino or 2-oxopyrimidin-1-yl radical and R₁₇ represents an alkyl radical.

22. The method of claim 20, wherein the method is effective to increase the luster, sheen, brilliance, gloss, glow, shine or patina of hair.
